(19) United States
(12) Patent Application Publication
(10) Pub. No.: US 2012/0010213 A1
(43) Pub. Date: Jan. 12, 2012

(54) ORAL CONTROLLED RELEASE DOSAGE FORMS FOR WATER SOLUBLE DRUGS

(75) Inventors: Mohan Anand CHANDAVARKAR, Bandra (IN); Kour Chand Jindal, Mumbai (IN); Rajkumar Malayandi, Tripur (IN)

(73) Assignee: FDC LIMITED, Mumbai (IN)

(21) Appl. No.: 13/225,984

(22) Filed: Sep. 6, 2011

Related U.S. Application Data
(63) Continuation of application No. PCT/IN2010/000121, filed on Mar. 2, 2010.

(30) Foreign Application Priority Data
Mar. 4, 2009 (IN) 468/MUM/2009

(51) Int. Cl. A61K 31/495; A61K 31/295; A61K 31/155; A61K 31/138; A61P 25/08; A61P 25/24; A61P 9/10; A61P 25/20; A61P 25/06; A61P 31/12; A61P 31/00; A61P 35/00; A61P 11/02; B29C 43/02; A61K 31/35

(52) U.S. Cl. 514/252.12; 514/455; 514/502; 514/635; 514/654; 514/646; 264/238

(57) ABSTRACT

Disclosed herein is an oral controlled release pharmaceutical formulation comprising water-soluble drug or pharmaceutically acceptable salts thereof, in a hydrophilic matrix system, further comprising pH independent polymers present in an amount of 5% to 90% w/w in combination with acid insoluble polymer present in an amount of 1% to 70% w/w and/or a diluent, a lubricant and/or a glidant.

This is a continuation of international patent application Ser. No. PCT/IN2010/000121, filed Mar. 2, 2010, published as WO 2010/100657.

### TECHNICAL FIELD

The present disclosure relates to a novel oral controlled release drug delivery system for water soluble drugs and their pharmaceutically acceptable salts thereof selected from therapeutic categories like neurotherapeutic agents, cardiovascular drugs, anti-infective, analgesics and drugs acting on endocrine and respiratory systems and a process of manufacturing the same.

### BACKGROUND

Developing oral controlled release matrix system for water-soluble drugs with constant release rate has always been a challenge to the pharmaceutical technologist. Most water-soluble drugs, if not formulated properly, may readily release the drug at a faster rate, and are likely to produce high blood concentrations leading to toxic effects after oral administration. The release of drug from controlled release dosage form not only determines the duration of therapeutic efficacy but also the toxicity. Design of a reliable and reproducible dosage form, which releases the drug in pre-programmed manner without the possibilities of dose dumping, is a challenging task and hence the development of such an ideal dosage form is essential to meet never ending medical demands.

Soluble drugs are difficult to formulate into a controlled release dosage forms. Solubility is a driving force for a drug substance to dissolve in water; the greater the solubility the greater the rate of dissolution, when all other factors are kept constant.

Large numbers of controlled release drug delivery systems have been reported in the literature. These systems are based on different drug release mechanisms such as diffusion controlled, disintegration controlled, osmotically controlled and pH triggered systems. However, many systems fail to produce desirable drug release in in-vivo environment. The ideal system should provide consistent release profile in both in-vitro and in-vivo condition and the drug release should be independent of physiological variability such as pH, gastrointestinal fluid and gastrointestinal transit time. Designing and development of controlled release or sustained release drug delivery system for the drugs having high aqueous solubility along with high dose is a challenging task for pharmaceutical scientist. Drugs having high solubility along with high dose easily leach from the matrix system and hence it is very difficult to control the release profile of the drug. Diffusion controlled release system using polymeric system have been used to deliver the soluble drugs.

Usage of water in wet granulation may interfere with stability of moisture sensitive drugs in dosage forms. Moreover, use of other solvents as wet granulation fluid, needs drying, monitoring of residual solvents, monitoring of toxicity of solvent and other environmental factors. The above constraints have been overcome by the present disclosure.

The present disclosure offers a technology which provides controlled mode of drug release of water soluble drugs in about 6 to 21 hours. Moreover, the dosage form is based on diffusion and erosion controlled release mechanism, predominantly, drug release is controlled by diffusion. Such release pattern offers uniform and desirable amount of plasma drug concentration. Another commonly associated problem with water soluble drugs is ‘burst release’ of drug from dosage form, leading to poor matrix mechanical stability and dose dumping. Dose dumping not only offers toxic plasma drug concentration but also leads to therapeutic failure. The present technology relates to unique combination of high molecular weight polymers and acid insoluble polymers that leads to better matrix integrity and minimum possibilities of dose dumping. The unique system also offers programmable controlled release profile of drug in biological fluids with better therapeutic efficacy.

### SUMMARY

In accordance with the above objectives, the present disclosure describes a novel controlled release oral pharmaceutical composition of water-soluble drugs and pharmaceutically acceptable salts thereof selected from therapeutic categories like cardiovascular drugs, neurotherapeutic agents, chemotherapeutic agents and drugs acting on endocrine system and a process of manufacturing the same in a hydrophilic matrix system, wherein the matrix comprises of non-enteric polymers in combination with acid enteric polymers, a diluent, a lubricant and a glidant.

According to various embodiments, the present disclosure describes an oral controlled release pharmaceutical formulation comprising a water-soluble drug or pharmaceutically acceptable salt thereof dispersed in a homogeneous hydrophilic matrix. In various embodiments, the hydrophilic matrix comprises at least two polymers having different solubility characteristics. At least one polymer in the matrix is a non-enteric polymer present in an amount of 5% to 90% by weight of the pharmaceutical formulation. At least one of the remaining polymers in the matrix is an enteric polymer which is insoluble in acid, but soluble in fluids having a neutral or basic pH. The enteric polymer is present in an amount of 1% to 70% by weight. Unlike enteric-coated dosage forms of the prior art, which do not release drugs in an acidic environment, the pharmaceutical formulation of the current disclosure releases a water-soluble drug or pharmaceutically acceptable salt thereof gradually over a period of at least 24 hours upon exposure to either an acidic environment or a non-acidic environment. The enteric polymer controls release in an acidic environment, while the non-enteric polymer controls release in a non-acidic environment.

In the current specification, the term “pH dependent polymer” refers to an enteric polymer, or to a polymer which is soluble or swellable in neutral or basic solution, but which is insoluble in acidic solution. Such polymers are, for example, insoluble in gastric fluid but soluble or swellable in neutral or basic fluids, such as intestinal fluid. The term “acid insoluble polymer,” as used in this specification, also refers to an enteric or pH dependent polymer. For purposes of this application, polymers which have a much greater solubility or swellability in neutral or basic conditions than in acidic conditions will be considered to be encompassed by the term enteric. Thus, for example, polycarbophil and its salts are considered to be enteric polymers. Polycarbophil, i.e., polyacrylic acid cross-linked with divinyl glycol. absorbs about ten times its own weight of water under acidic conditions prevailing in the stomach, but absorbs 70 times its weight under pH-neutral conditions.

The terms “non-enteric polymer” and “pH independent polymer” are here understood to refer to a polymer which is non-enteric, i.e., which is not more soluble in non-acidic media than in acidic media. The terms “non-enteric polymer” and “pH independent polymer” therefore encompass polymers which are equally soluble in acidic, and neutral or basic media. The terms “non-enteric polymer” and “pH independent polymer,” as used in this disclosure, may additionally encompass polymers which are more soluble in acidic media than in neutral or basic media.

In certain embodiments, the oral controlled release pharmaceutical formulation comprises a water-soluble drug or pharmaceutically acceptable salt thereof dispersed in a homogeneous hydrophilic matrix formed from at least one non-enteric polymer; at least one enteric polymer; and an optional additive selected from the group consisting of a diluent, a lubricant, and a glidant.

Various embodiments relate to an oral controlled release pharmaceutical formulation prepared by blending a water-soluble drug or pharmaceutically acceptable salt thereof, at least one non-enteric polymer, and at least one enteric polymer to produce a homogeneous blend; optionally granulating the blend with a granulating solvent; optionally combining the blend with a lubricant; and preparing an oral dosage form from the blend by direct compression.

The present disclosure, according to various embodiments, provides a novel controlled release oral pharmaceutical composition of water-soluble drugs and pharmaceutically acceptable salts thereof selected from therapeutic categories like cardiovascular drugs, neurotherapeutic agents, chemotherapeutic agents and drugs acting on the endocrine system. The present disclosure further provides a process of manufacturing the same in a hydrophilic matrix system, wherein the matrix comprises of pH independent, i.e., non-enteric, polymers in combination with acid insoluble, i.e., enteric, polymers, an optional diluent, an optional lubricant and an optional glidant.

### DETAILED DESCRIPTION

Various embodiments will now be described in details in connection with certain preferred and optional embodiments so that various aspects thereof may be more fully understood and appreciated.

In this specification, the term “acidic pH” refers to a pH of less than about 5, preferably a pH of less than about 3, corresponding to pH of gastric fluids in the stomach.

In this specification, the term “neutral pH” corresponds to a pH value of about 5 to about 8. The term “basic pH” corresponds to a pH value of greater than about 8. Since the mean pH of the duodenum is between about 5 to about 6, and the pH of the small intestine is about 8, the term “neutral or basic pH” corresponds to a pH greater than 5, and describes the environment within either the duodenum or the small intestine.

Various embodiments describe an oral controlled release pharmaceutical formulation comprising water-soluble drugs or pharmaceutically accepted salts thereof in a hydrophilic matrix system by direct compression or granulation.

Various embodiments include a hydrophilic matrix system composed of at least one pH independent polymer in combination with at least one pH dependent or enteric polymer, along with pharmaceutically acceptable excipients.

The active ingredient in the formulation is a water soluble drug or a salt thereof, which may be selected from the group consisting of:
cardiovascular drugs selected from the group consisting of antilipedemics, β-blockers, ACE inhibitors, diuretics, α-receptor agonists, calcium channel blockers, anticoagulants, antianginal and anti arrhythmic agents, and mixtures thereof;
neurotherapeutic agents selected from the group consisting of antiepileptics, antidepressants, tranquillizers, psychotherapeutic agents, sedatives and hypnotics, antimigraine agents, antipyretic agents, antiemetics, and antispasmodic agents, and mixtures thereof;
antiinfective agents selected from the group consisting of β lactam antibiotics, macrolide antibiotics, antifungal agents, antiviral agents, and cytotoxic chemotherapeutic agents, and mixtures thereof;
drugs acting on the endocrine system selected from the group consisting of oral hypoglycemic agents, thyroid and antithyroid drugs, synthetic and semi synthetic hormones, and mixtures thereof;
drugs acting on respiratory system selected from the group consisting of antitussives, decongestants and anti-asthmatics, and mixtures thereof; and
mixtures thereof.

The active ingredient may be present in the formulation in an amount of between about 1% and about 80% by weight of the formulation, preferably from about 5% to about 70% by weight.

In various embodiments, the present disclosure provides a hydrophilic matrix system comprising non-enteric polymers in combination with enteric polymers which are insoluble in acid, but soluble in neutral or basic solutions. The matrix system is homogeneous, and comprises non-enteric polymers, enteric polymers, and optional additives. These additives include diluents, lubricants and/or glidants. The matrix system provides controlled drug release in the absence of an additional release-controlling coating.

In various embodiments, non-enteric polymers are incorporated in the formulation to provide release of water soluble drugs from the formulation in an acidic environment.

In certain embodiments, non-enteric polymers are defined as polymers which are erodible, dispersible, or soluble in an acidic environment, such as the stomach. These polymers may exhibit pH-independent solubility, i.e., they may be soluble or dispersible in both acid and base. Alternatively, non-enteric polymers may be more soluble in acidic environments, i.e., the stomach, than in neutral or basic environments, i.e., the small intestine. If the non-enteric polymers are soluble in acidic environments, the non-enteric polymers may be lightly crosslinked to produce an insoluble polymer which is water-swellable under acidic pH conditions. This allows water to enter the matrix and dissolve the water-soluble drug, while helping to maintain matrix entegrity.

Non-enteric polymers may be selected from the group consisting of cellulose derivatives, chitosan derivatives, natural gums, polymethacrylates, and mixtures thereof. Cellulose derivatives may be selected from the group consisting of Hydroxypropyl Cellulose, Hydroxypropyl ethyl cellulose, Hydroxyethyl Cellulose, Hydroxypropylmethyl cellulose and Hydroxymethyl Cellulose; preferably Hydroxyethyl Cellulose and Hydroxypropylmethyl cellulose; most preferably Hydroxypropylmethyl cellulose. The non-enteric polymers are present in the formulation in an amount ranging from about 5% by weight of the formulation to about 90% by weight of the formulation, preferably about 10% to about 60%.

The hydrophilic matrix contains enteric polymers in a substantially homogeneous mixture with the non-enteric polymers. The enteric polymers, unlike the non-enteric polymers, are not erodible, dispersible, or soluble in an acidic environment. Enteric polymers are used to retard the release of drug in stomach. Additionally, they prevent the tablet from breaking up in the stomach by providing matrix integrity to the tablet. By retarding drug release in the stomach, the enteric polymers help prevent dose dumping.

The stomach contains a high volume of acidic fluid of a low viscosity. Additionally, the acidic fluids in the stomach undergo mixing by the stomach. The small intestine, in contrast, contains a neutral or mildly basic fluid of high viscosity. Due to the high volume and low viscosity of stomach contents, water soluble drugs have increased solubility and undergo more rapid dissolution in the stomach, when compared to solubility and dissolution rate of such drugs in the highly viscous fluids of the proximal part of the gastrointestinal tract, i.e., the small intestine.

In the absence of an enteric polymer, a water soluble drug is rapidly released from a non-enteric matrix polymer in the stomach. Gastric mixing leads to rapid disintegration of the polymer matrix in such conditions. Additionally, the non-enteric matrix polymer undergoes dissolution, dispersion, or erosion in the stomach, allowing rapid dissolution of the water-soluble drug from the tablet matrix. The disintegration of non-enteric polymer matrices, together with rapid drug dissolution, results in dose dumping. Dose dumping is undesirable, because it can result in toxic plasma drug concentration immediately after drug release, and/or plasma drug concentrations below therapeutic levels subsequent to the initial release of the drug.

An enteric polymer does not undergo substantial dissolution, dispersion, or erosion in the stomach. Use of an enteric polymer in the absence of a non-enteric polymer substantially prevents any drug release in the acidic environment of the stomach. Enteric polymers therefore prevent any drug release until the dosage form passes into the neutral or basic environment of the small intestine. The enteric polymer is dissolved or dispersed rapidly in an acidic or neutral environment, leading to rapid release of drug in the small intestine.

Unlike either an enteric polymer or a non-enteric polymer individually, a polymer matrix comprising a homogeneous blend of an enteric polymer and a non-enteric polymer allows gradual release of a water-soluble drug in both the acidic environment of the stomach and the basic or neutral environment of the small intestine. Non-enteric polymers may undergo swelling or dispersion in the stomach, allowing gastric fluids to begin dissolving the water-soluble drug. However, the enteric polymers do not dissolve or disperse in the stomach, and hence maintain the integrity of the polymer matrix and retard dissolution of the drug. By retarding drug dissolution, enteric polymers help prevent dose dumping in the stomach. Instead, use of a polymer matrix comprising an enteric polymer and a non-enteric polymer results in gradual drug release in the stomach.

According to various embodiments, the pharmaceutical formulation provides gradual drug release over an extended period, i.e., a period of at least 24 hours, in an acidic environment. The pharmaceutical formulation also provides gradual drug release over an extended period, i.e., a period of at least 24 hours, in an acidic environment. According to various embodiments, extended release may be achieved in the absence of a release-controlling coating surrounding the polymer matrix. In various embodiments, coatings which do not provide extended release, i.e., coatings comprising polymers which rapidly dissolve or disperse in the mouth or in the stomach, i.e., polyethylene glycols, hydroxypropylmethyl cellulose, etc., may be provided surrounding the polymer matrix. Suitable coating materials include materials available from Colorcon® under the Opadry® trade name.

When the dosage form reaches the proximal part of the gastrointestinal tract (GIT), i.e., the duodenum or the small intestine, the enteric polymer, or acid insoluble and base soluble polymer, in the hydrophilic matrix polymers are dissolved or dispersed in the intestinal content. This exposes drug particles within the polymer matrix to intestinal fluids, and offers a large surface area for drug dissolution and drug release from the polymer matrix. At the same time, high molecular weight non-enteric polymers remaining in the polymer matrix dissolve or swell in the viscous intestinal fluids, forming a viscous gel around the drug particles. Release of the water soluble drug in the small intestine is retarded by this viscous gel, resulting in a gradual release of drug in the intestine. Thus, the release profile of the water soluble drug is controlled by the combination of enteric and non-enteric polymers, where enteric polymers act to retard drug release in the stomach, while non-enteric polymers act to retard drug release in the proximal part of the gastrointestinal tract, i.e., the duodenum or the small intestine. Depending on the pKa of the enteric polymer, the enteric polymer may dissolve or disperse in the duodenum (pH 5-6.5) or in the small intestine (pH 7-9).

In various embodiments, the enteric polymers are selected from group of Carbopol®, alginic acid, salts of alginic acid and their derivatives, polymers of acrylic acid derivatives, and phthalates, acetates, succinates or acetate succinates of cellulose esters, preferably polymers of acrylic acid derivatives and Carbopol®, most preferably polymers of methacrylic acid derivatives. The enteric polymers are present in the formulation in amounts ranging from about 1% to about 70% w/w, preferably about 5% to about 50% w/w. Preferred enteric polymers include polymers sold under the trade name EUDRAGIT®. Suitable EUDRAGIT® polymers are anionic polymers of methacrylic acid and methacrylates. They contain —COOH as a functional group. They dissolve at ranges from pH 5.5 to pH 7, i.e., pH values which are less acidic than gastric juice, but which roughly correspond to pH values in the duodenum and/or the small intestine.

A diluent is selected from the group consisting of microcrystalline cellulose, powdered cellulose, lactose, sorbitol, mannitol, sucrose, mannose, galactose, anhydrous calcium phosphates such as mono, di and tri basic preferably microcrystalline cellulose and dibasic calcium phosphate. The diluent in the formulation present in an amount ranging from about 1% to about 95% w/w, preferably about 5% to about 80% w/w.

A lubricant is selected from the group consisting of magnesium stearate, calcium stearate, glyceryl monostearate, glyceryl palmitostearate, stearic acid, talc, zinc stearate, magnesium lauryl sulfate and colloidal silicon dioxide, preferably magnesium stearate. The lubricant in the formulation is ranging from about 0.1% to about 10% w/w and preferably about 0.2% to about 5.0% w/w.

Glidants are added to improve the flow properties of the formulation and to improve the accuracy of dosing. The glidant is selected from the group consisting of silicon dioxide, magnesium trisilicate, powdered cellulose, starch, talc and tribasic calcium phosphate preferably colloidal silicon dioxide. The glidant is present in the formulation in an amount ranging from about 0.25% to about 5.0% w/w.

According to various embodiments, an oral controlled release delivery system for water soluble drugs and their pharmaceutical acceptable salt thereof are prepared by direct compression or granulation method in a hydrophilic pH independent matrix system using pH independent polymer in combination with acid insoluble polymer. For moisture sensitive drugs, the use of water in wet granulation may interfere with drug stability in dosage forms. Moreover, other solvents used wet granulation fluid require drying, and may create issues with the final dosage form. These issues include the presence of residual solvents, including residual solvents which may be toxic, and other environmental issues. Direct compression is not only the most convenient process for making a stable formulation, but also provides cost effective formulations.

In various embodiments, the composition according to the present disclosure comprises the following components:
Active (1-80% w/w, preferably 5-70% w/w)
Acid insoluble polymer (20-70% w/w)
Matrix forming agent or pH independent polymer(10-90% w/w)
Diluents (0-95% w/w, preferably 5-95% w/w, more preferably 10-70 w/w)
Lubricants (qs)
Glidants (qs)

The following exemplary embodiments will serve to illustrate the disclosed subject matter, it being understood that the particulars shown are by way of example and for purpose of illustrative discussion of certain embodiments of the invention.

### EXAMPLES

### Example 1

Accurately weighted quantities of Topiramate, hydroxypropylmethyl cellulose, Eudragit®, cellulose, microcrystalline and colloidal silicon dioxide are sieved individually and are blended thoroughly. The blend is lubricated with Magnesium Stearate and directly compressed using 9.5 mm S.C. punches. The ingredients are added in the following proportions.

| | | |
|---|---|---|
| Topiramate | 200 | mg |
| Hydroxypropylmethyl | 50 | mg |
| cellulose | | |
| Eudragit ® | 50 | mg |
| Microcrystalline Cellulose | 100 | mg |
| Magnesium Stearate | 3 | mg |
| Colloidal Silicon Dioxide | 3 | mg |

In vitro dissolution profile

| | % drug dissolution | | | |
|---|---|---|---|---|
| Time | | | 6.8 phosphate | 7.4 phosphate |
| (hrs) | 0.1N HCl | Water | buffer | buffer |
| 0.25 | 10 | 14 | 17 | 17 |
| 0.5 | 13 | 19 | 21 | 22 |
| 1 | 17 | 23 | 30 | 27 |
| 3 | 27 | 33 | 32 | 43 |
| 6 | 33 | 47 | 47 | 54 |
| 9 | 36 | 64 | 49 | 60 |
| 12 | 38 | 79 | 59 | 70 |
| 24 | 51 | 93 | 83 | 90 |

### Example 2

Accurately weighted quantities of Topiramate, hydroxypropylmethyl cellulose, sodium alginate and microcrystalline cellulose are sieved individually and are blended thoroughly and granulated with IPA: water 70:30. The blend was lubricated with magnesium stearate and colloidal silicon dioxide before compression using 9.5 mm S.C. punches. The ingredients are added in the following proportions.

| | | |
|---|---|---|
| Topiramate | 200 | mg |
| Hydroxypropylmethyl | 50 | mg |
| cellulose | | |
| Sodium Alginate | 50 | mg |
| Microcrystalline Cellulose | 100 | mg |
| Magnesium Stearate | 3 | mg |
| Colloidal Silicon Dioxide | 3 | mg |

In vitro dissolution profile

| Time | % drug dissolution | |
|---|---|---|
| (hrs) | 0.1N HCl | Water |
| 0.25 | 5 | 4 |
| 0.5 | 7 | 13 |
| 1 | 8 | 28 |
| 3 | 12 | 55 |
| 6 | 17 | 82 |
| 9 | 20 | 92 |
| 12 | 23 | 93 |
| 24 | 26 | 96 |

### Example 3

Accurately weighted quantities of sodium feredetate, hydroxypropylmethyl cellulose, polycarbophil, anhydrous dibasic calcium phosphate and colloidal silicon dioxide are sieved individually and are blended thoroughly. The blend was lubricated with magnesium stearate and directly compressed using 9.5 mm S.C. punches. The ingredients are added in the following proportions.

| | | |
|---|---|---|
| Sodium Feredetate | 257 | mg |
| Hydroxypropylmethyl | 50 | mg |
| cellulose | | |
| Polycarbophil | 50 | mg |
| Anhydrous Dibasic Calcium | 100 | mg |
| Phosphate | | |
| Magnesium Stearate | 3 | mg |
| Colloidal Silicon Dioxide | 3 | mg |

In vitro dissolution profile

| Time | % drug dissolution | |
|---|---|---|
| (hrs) | 0.1N HCl | Water |
| 0.25 | 8 | 15 |
| 0.5 | 14 | 29 |
| 1 | 16 | 38 |
| 3 | 21 | 56 |
| 6 | 24 | 68 |
| 9 | 33 | 73 |
| 12 | 38 | 85 |
| 24 | 49 | 97 |

### Example 4

Accurately weighted quantities of metformin, hydroxypropylmethyl cellulose, microcrystalline cellulose and Eudragit® are sieved individually and are blended thoroughly and granulated with IPA: water 70:30. The blend was lubricated with Magnesium Stearate and Colloidal Silicon Dioxide before compression using 21.0×10.5 mm S.C. punches. The ingredients are added in the following proportions.

| | | |
|---|---|---|
| Metformin | 1000 | mg |
| Hydroxypropylmethyl | 200 | mg |
| cellulose | | |
| Eudragit ® | 200 | mg |
| Microcrystalline Cellulose | 150 | mg |
| Magnesium Stearate | 10 | mg |
| Colloidal Silicon Dioxide | 10 | mg |

### Example 5

Accurately weighted quantities of metformin, hydroxypropyl cellulose, hydroxypropylmethyl cellulose, microcrystalline cellulose and Eudragit® are sieved individually and are blended thoroughly and granulated with water. The blend was lubricated with magnesium stearate and colloidal silicon dioxide before compression using 21.0×10.5 mm S.C. punches. The ingredients are added in following proportions.

| | | |
|---|---|---|
| Metformin | 1000 | mg |
| Hydroxypropyl Cellulose | 100 | mg |
| Hydroxypropylmethyl | 100 | mg |
| cellulose | | |
| Eudragit ® | 200 | mg |
| Microcrystalline Cellulose | 100 | mg |
| Magnesium Stearate | 10 | mg |
| Colloidal Silicon Dioxide | 10 | mg |

### Example 6

Accurately weighted quantities of venlafaxine hydrochloride, hydroxypropylmethyl cellulose, Eudragit® and microcrystalline cellulose are sieved individually and are blended thoroughly and granulated with IPA. The blend was lubricated with magnesium stearate and colloidal silicon dioxide before compression using 8.0 mm S.C. punches. The ingredients are added in following proportions.

| | | |
|---|---|---|
| Venlafaxine hydrochloride | 150 | mg |
| Hydroxypropylmethyl | 100 | mg |
| cellulose | | |
| Eudragit ® | 50 | mg |
| Microcrystalline Cellulose | 200 | mg |
| Magnesium Stearate | 5 | mg |
| Colloidal Silicon Dioxide | 3 | mg |

### Example 7

Accurately weighted quantities of trimetazidine, hydroxypropyl cellulose, sodium alginate, microcrystalline cellulose and colloidal silicon dioxide are sieved individually and are blended thoroughly. The blend was lubricated with magnesium stearate and directly compressed using 7.0 mm S.C. punches. The ingredients are added in following proportions.

| | | |
|---|---|---|
| Trimetazidine | 60 | mg |
| Hydroxypropyl Cellulose | 100 | mg |
| Sodium Alginate | 100 | mg |
| Microcrystalline Cellulose | 100 | mg |
| Magnesium Stearate | 5 | mg |
| Colloidal Silicon Dioxide | 3 | mg |

### Example 8

Accurately weighted quantities of tramadol hydrochloride, hydroxypropyl cellulose, carbopol, microcrystalline cellulose and colloidal silicon dioxide are sieved individually and are blended thoroughly. The blend was lubricated with magnesium stearate and directly compressed using 9.5 mm S.C. punches. The ingredients are added in following proportions.

| | | |
|---|---|---|
| Tramadol hydrochloride | 200 | mg |
| Hydroxypropylmethyl | 50 | mg |
| cellulose | | |
| Carbopol | 100 | mg |
| Microcrystalline Cellulose | 100 | mg |
| Magnesium Stearate | 4 | mg |
| Colloidal Silicon Dioxide | 3 | mg |

We claim:

1. An oral controlled release pharmaceutical formulation comprising a water-soluble drug or pharmaceutically acceptable salt thereof dispersed in a homogeneous hydrophilic matrix, wherein said hydrophilic matrix comprises:
at least one non-enteric first polymer present in an amount of about 5% to about 90% by weight,
at least one second polymer present in an amount of about 1% to about 70% by weight, and
an optional additive selected from the group consisting of a diluent, a lubricant, and a glidant;
wherein said second polymer is insoluble in acid, but soluble or dispersible at neutral or basic pH;
wherein said pharmaceutical formulation releases said water-soluble drug or pharmaceutically acceptable salt thereof gradually over a period of at least 24 hours upon exposure to either an acidic environment or a non-acidic environment.

2. The oral controlled release pharmaceutical formulation as claimed in claim 1, wherein said water soluble drug is selected from the group consisting of cardiovascular drugs, antilipedemics, β-blockers, ACE inhibitors, diuretics, α-receptor agonists, calcium channel blockers, anticoagulants, antianginal agents, antiarrhythmic agents, antiepileptics, antidepressants, tranquillizers, psychotherapeutic agents, sedatives, hypnotics, antimigraine agents, antipyretic agents, antiemetics, antispasmodic agents, β-lactam antibiotics, macrolide antibiotics, antifungal agents, antiviral agents, antifungal agents, chemotherapeutic agents, oral hypoglycemic agents, thyroid and antithyroid drugs, synthetic and semisynthetic hormones, antitussives, decongestants and antiasthmatics.

3. The oral controlled release pharmaceutical formulation as claimed in claim 1, wherein said pharmaceutical formulation contains from about 1% to about 80% by weight of said water soluble drug.

4. The oral controlled release pharmaceutical formulation as claimed in claim 1, wherein said first polymer is selected from the group consisting of:
a cellulose derivative selected from the group consisting of hydroxypropyl cellulose, hydroxypropyl ethyl cellulose, hydroxyethyl cellulose, hydroxypropylmethyl cellulose, and hydroxymethyl cellulose;
a chitosan derivative;
a natural gum;
a polymethacrylate; and
a mixture thereof.

5. The oral controlled release pharmaceutical formulation as claimed in claim 1, wherein said second polymer is selected from the group consisting of:
phthalates, acetates, succinates and acetate succinate of cellulose esters,
Carbopol, acrylic acid derivatives
alginic acid,
salts and derivatives of alginic acid, and
acid-insoluble polymers of (meth)acrylic acid and derivatives thereof.

6. The oral controlled release pharmaceutical formulation as claimed in claim 1, wherein said diluent is selected from the group consisting of microcrystalline cellulose, powdered cellulose, lactose, sorbitol, mannitol, sucrose, mannose, galactose, and anhydrous calcium phosphate,
wherein said calcium phosphate is monobasic, dibasic, or tribasic.

7. The oral controlled release pharmaceutical formulation as claimed in claim 1, wherein said lubricant is selected from the group consisting of magnesium stearate, calcium stearate, glyceryl monostearate, glyceryl palmitostearate, stearic acid, talc, zinc stearate, magnesium lauryl sulfate and colloidal silicon dioxide.

8. The oral controlled release pharmaceutical formulation as claimed in claim 1, wherein said glidant is selected from the group consisting of colloidal silicon dioxide, fumed silicon dioxide, magnesium trisilicate, powdered cellulose, starch, talc and tribasic calcium phosphate.

9. An oral controlled release pharmaceutical formulation, wherein said controlled release formulation is prepared by:
blending a water-soluble drug or pharmaceutically acceptable salt thereof, at least one pH independent first polymer, and at least one second polymer to produce a homogeneous blend;
optionally granulating said blend with a granulating solvent;
optionally combining said blend with a lubricant; and
preparing an oral dosage form from said blend by direct compression;
wherein said second polymer is insoluble in acid, but soluble in intestinal fluid.

10. The oral controlled release pharmaceutical formulation of claim 9, wherein said blending step comprises blending:
a water-soluble drug or pharmaceutically acceptable salt thereof,
at least one pH independent first polymer,
at least one second polymer, and
an optional additive selected from the group consisting of a diluent, a lubricant, and a glidant.

11. An oral controlled release pharmaceutical formulation comprising a water-soluble drug or pharmaceutically acceptable salt thereof dispersed in a homogeneous hydrophilic matrix, wherein said hydrophilic matrix comprises:
at least one first polymer selected from the group consisting of hydroxypropyl cellulose, hydroxypropyl ethyl cellulose, hydroxyethyl cellulose, hydroxypropylmethyl cellulose, and hydroxymethyl cellulose, said first polymer being present in an amount of 5% to 90% by weight,
at least one enteric polymer selected from the group consisting of a copolymer of methacrylic acid and methacrylates, sodium alginate, and polycarbophil, said enteric polymer being present in an amount of about 1% to about 70% by weight, and
an optional additive selected from the group consisting of a diluent, a lubricant, and a glidant;
wherein said pharmaceutical formulation releases said water-soluble drug or pharmaceutically acceptable salt thereof gradually over a period of at least 24 hours upon exposure to either an acidic environment or a non-acidic environment.

12. An oral controlled release pharmaceutical formulation comprising a water-soluble drug or pharmaceutically acceptable salt thereof dispersed in a homogeneous hydrophilic matrix, wherein said hydrophilic matrix comprises:
at least one non-enteric first polymer present in an amount of about 5% to about 90% by weight,
at least one second polymer present in an amount of about 1% to about 70% by weight, and
an optional additive selected from the group consisting of a diluent, a lubricant, and a glidant;
wherein said second polymer is insoluble in acid, but soluble or dispersible at neutral or basic pH;
wherein said pharmaceutical formulation does not include a release-controlling coating.

13. An oral controlled release pharmaceutical formulation comprising:
a homogeneous hydrophilic matrix system;
at least one water-soluble drug or pharmaceutically acceptable salt thereof dispersed in said matrix system; and
an optional additive selected from the group consisting of a diluent, a lubricant a glidant, and mixtures thereof, said additive being dispersed in said matrix system;
wherein said matrix system comprises a homogeneous blend of:
at least one pH independent polymer present in an amount of 5% to 90% w/w; and
at least one acid insoluble polymer present in an amount of 1% to 70% w/w;
wherein said pharmaceutical formulation releases said water-soluble drug or pharmaceutically acceptable salt thereof gradually upon exposure to either an acidic environment or a non-acidic environment.
